(19) United States
(12) Patent Application Publication
(10) Pub. No.: US 2026/0215856 A1
(43) Pub. Date: Jul. 30, 2026

(54) OPHTHALMIC SURGERY ROBOTIC SYSTEM

(71) Applicant: Alcon Inc., Fribourg (CH)

(72) Inventors: Patrick Terry, Goleta, CA (US); Satish Yalamanchili, Irvine, CA (US); Nanhong Lou, Southlake, TX (US)

(21) Appl. No.: 19/455,003

(22) Filed: Jan. 21, 2026

Related U.S. Application Data
(60) Provisional application No. 63/749,977, filed on Jan. 27, 2025.

(51) Int. Cl. A61B 34/20; A61B 34/00; A61B 34/30; A61B 50/33

(52) U.S. Cl. CPC A61B 34/20; A61B 34/30; A61B 34/70; A61B 50/33; A61B 2034/2055

(57) ABSTRACT

In certain embodiments, a robotic system for a surgical facility includes a dispenser, a delivery robot, and a setup robot located in an operating room. The delivery robot includes a drive system, sensors, a robotic arm including a camera and a gripper, and a control system that is configured to navigate to the dispenser based on sensor data, retrieve a bin containing a surgical package from the dispenser, navigate to a storage table in the operating room based on the sensor data, and deliver the packages to the storage table. The setup robot includes a robotic arm including first and second end effectors, and a control system that is configured to open the surgical package located on the storage table, remove items from the surgical package, and deposit the items on a sterile tray in the operating room.

### CROSS-REFERENCE TO RELATED APPLICATIONS

This application claims the benefit of U.S. Provisional Application Ser. No. 63/749,977 (filed on January 27, 2025), the content of which is incorporated by reference herein in its entirety.

### BACKGROUND

The present disclosure relates to ophthalmic surgery, and, more particularly, to an ophthalmic surgery robotic system.

Common ophthalmic surgical procedures include cataract surgery, glaucoma treatments, retinal membrane peeling, vitrectomy, and retinal reattachment. The structures of the eye are extremely small and delicate. Ophthalmic surgery is therefore extremely sophisticated and becoming an ophthalmic surgeon requires many years of training. The time of an ophthalmic surgeon spent in the operating room is therefore a very valuable resource.

### SUMMARY

In certain embodiments, a robotic system for a surgical facility comprises a dispenser, a delivery robot, and a setup robot located in an operating room.

The delivery robot comprises a drive system, sensors configured to generate sensor data, a robotic arm comprising a camera and a gripper, and a control system configured to navigate to the dispenser based on the sensor data, retrieve a bin including a surgical package from the dispenser using the gripper, navigate to a storage table in an operating room based on the sensor data, and deliver the surgical package to the storage table using the gripper.

The setup robot comprises a robotic arm and a control system. The robotic arm comprises a camera, a first end effector configured to hold and manipulate a package, and a second end effector configured to open the package. The control system is configured to open the surgical package located on the storage table using the first and second end effectors, remove items from the surgical package using the first end effector, and deposit the items on a sterile tray in the operating room using the first end effector.

In certain embodiments, the robotic system for the surgical facility further comprises a storage robot located in a storage room adjacent to the dispenser. The storage robot comprises a drive system, sensors configured to generate sensor data, a robotic arm comprising a camera and a gripper, and a control system configured to navigate to a storage rack within the storage room based on the sensor data, retrieve one or more surgical packages from the storage rack using the gripper, navigate to a back side of the dispenser based on the sensor data, and deliver the surgical packages to a bin in one of the compartments of the dispenser using the second gripper.

### BRIEF DESCRIPTION OF THE DRAWINGS

FIG. 1 depicts a schematic diagram of a surgical facility for ophthalmic procedures, in accordance with embodiments of the present disclosure.

FIG. 2A depicts an elevation view of a dispenser for a surgical facility, in accordance with embodiments of the present disclosure.

FIG. 2B depicts a storage robot, in accordance with embodiments of the present disclosure.

FIG. 2C depicts a block diagram of the storage robot depicted in FIG. 2B, in accordance with embodiments of the present disclosure.

FIG. 2D depicts a schematic diagram of a storage room with another storage robot for a surgical facility, in accordance with embodiments of the present disclosure.

FIG. 2E depicts a block diagram of the storage robot depicted in FIG. 2D, in accordance with embodiments of the present disclosure.

FIG. 3A depicts another schematic diagram of the surgical facility for ophthalmic procedures, in accordance with embodiments of the present disclosure.

FIG. 3B depicts a diagram of a delivery robot, in accordance with embodiments of the present disclosure.

FIG. 3C depicts a block diagram of the delivery robot depicted in FIG. 3B, in accordance with embodiments of the present disclosure.

FIG. 4A depicts another schematic diagram of the surgical facility for ophthalmic procedures, in accordance with embodiments of the present disclosure.

FIG. 4B depicts a diagram of a setup robot, in accordance with embodiments of the present disclosure.

FIG. 4C depicts a block diagram of the setup robot depicted in FIG. 4B, in accordance with embodiments of the present disclosure.

FIG. 5A depicts another schematic diagram of the surgical facility for ophthalmic procedures, in accordance with embodiments of the present disclosure.

FIG. 5B depicts a diagram of a setup robot, in accordance with embodiments of the present disclosure.

FIG. 5C depicts a block diagram of the setup robot depicted in FIG. 5B, in accordance with embodiments of the present disclosure.

FIG. 6 depicts another schematic diagram of the surgical facility for ophthalmic procedures, in accordance with embodiments of the present disclosure.

FIG. 7 depicts a block diagram for a control system for the surgical facility for ophthalmic procedures, in accordance with embodiments of the present disclosure.

### DETAILED DESCRIPTION

In certain embodiments, a surgical facility for ophthalmic procedures may include a pre-operation (pre-op) room or holding area, a post-operation (post-op) room or holding area, an eye imaging room, a first operating room, a second operating room, a sterilization room, a storage room, a computer room, etc. The ophthalmic surgical facility may be arranged around a central hallway. The pre-op room and the post-op room may be located at opposite ends of the hallway. The first operating room, the second operating room, and the sterilization room may be located on one side of the hallway, and the eye imaging room, the storage room, and the computer room may be located on the other side of the hallway. Other arrangements, rooms, etc., are also supported, such as a single operating room, a hallway that is located along one side of the ophthalmic surgical facility, etc.

A patient may be prepared for ophthalmic surgery in the pre-op room. The patient is moved to a gurney in the pre-op room, transported to certain rooms in the ophthalmic surgical facility in a particular sequence, and then transported to the post-op room for recovery. In one example, a surgeon may perform an ophthalmic procedure on both eyes of the patient, such as cataract surgery. A nurse may transport the patient from the pre-op room into the hallway, and then into the eye imaging room. After high-resolution images, measurements, etc., of the eyes are captured by an imaging system, such as Alcon's LenSx^{®}system, etc., the nurse transports the patient to the first operating room. After the surgeon performs the ophthalmic procedure on the first eye (such as the right eye), the nurse may transport the patient to the second operating room. After the surgeon performs the ophthalmic procedure on the second eye (such as the left eye), the nurse may transport the patient to the post-op room. In another example, a surgeon may perform an ophthalmic procedure on one eye of the patient, such as the right eye. In this example, high-resolution images, measurements, etc., of the eye are captured by the imaging system, and, after the surgeon performs the ophthalmic procedure on the eye in the first or second operating room, the nurse may transport the patient to the post-op room.

Each operating room may include a surgical microscope mounted to an adjustable support, a surgical console with a visualization system and a phacoemulsification (phaco) handpiece (such as Alcon's Centurion® Vision System with Active Sentry® handpiece), a sterile table configured to support a sterile tray with items that will be used during the ophthalmic procedure, a disposal station for disposing, storing or processing used items from the sterile tray. The items needed for the ophthalmic procedures may include reusable instruments (such as phaco handpieces, etc.), consumable products contained in surgical packs or packages, structures to be implanted (such as intraocular lenses (IOLs), etc.), etc. Certain items arrive at the surgical facility in sealed packages to protect the sterility of each item prior to the ophthalmic procedure, such as consumable products, IOLs, etc.

Before the ophthalmic procedures begin each day, surgical staff retrieve the items that will be used during the day from the storage room (such as IOL and surgical packages containing one or more items, etc.) and the sterilization room (such as reusable instruments enclosed in a sterile package, etc.), and stage the packages for each ophthalmic procedure on a staging table outside each operating room. Before each ophthalmic procedure, surgical staff retrieve the packages for that ophthalmic procedure from the staging table outside the operating room, place the packages on the storage table in the operating room, open each package, and place the items and instruments on the sterile tray on the sterile table.

Initially staging the packages on staging tables, and then retrieving the packages from the staging tables, opening the packages, and arranging the items from the packages on the sterile tray is time consuming and labor intensive. Additionally, the required motions that surgical staff perform when opening these sealed packages are technically complex, and surgical staff training bottlenecks may prevent the optimum efficiency. Furthermore, the inventory of the items placed on the sterile trays is not automatically tracked, and may be dependent upon the diligence of the surgical staff. Because high-volume ophthalmic procedure facilities require a large number of surgical staff, staffing shortages for nurses, scrub technicians, etc., impact the efficiency of these facilities. Staffing shortages invariably lead to the need for surgical staff to work overtime hours, which causes stress and high mental strain.

Embodiments of the present disclosure advantageously provide a robotic system for a surgical facility that performs certain activities of the surgical staff at the beginning of the day and before each ophthalmic procedure, which reduces surgical staff requirements and duties, does not require training of new surgical staff or re-training of current surgical staff, automatically tracks inventory, etc.

In certain embodiments, the robotic system for a surgical facility may include a dispenser that is configured to store IOL and surgical packages, a setup robot that is located in an operating room, and a delivery robot that navigates between the dispenser and the operating room. The delivery robot may be configured to retrieve the IOL and surgical packages from the dispenser, and deliver them to a storage table in the operating room. The setup robot may be configured to open the IOL and surgical packages, and arrange the items on the sterile tray.

When the surgical facility has multiple operating rooms, the delivery robot may navigate between the dispenser and each operating room, and the setup robot may navigate between the operating rooms. In some embodiments, a setup robot may be located in each operating room. When the surgical facility has a sterilization room, the delivery robot may also navigate between the sterilization room and the operating room(s) in order to retrieve reusable instruments. Additionally, the dispenser may be located adjacent to a storage room, and a storage robot may be located in the storage room. The storage robot may be configured to fill the dispenser with the IOL and surgical packages that are needed for each ophthalmic procedure to be performed that day.

Other configurations of the robotic system are also supported. In some embodiments, the surgical facility may include the dispenser and the storage robot, the delivery robot, or the setup robot. In some embodiments, the surgical facility may include the dispenser, the storage robot, and the delivery robot. In some embodiments, the surgical facility may include the dispenser, the storage robot, the delivery robot, and one or more setup robots.

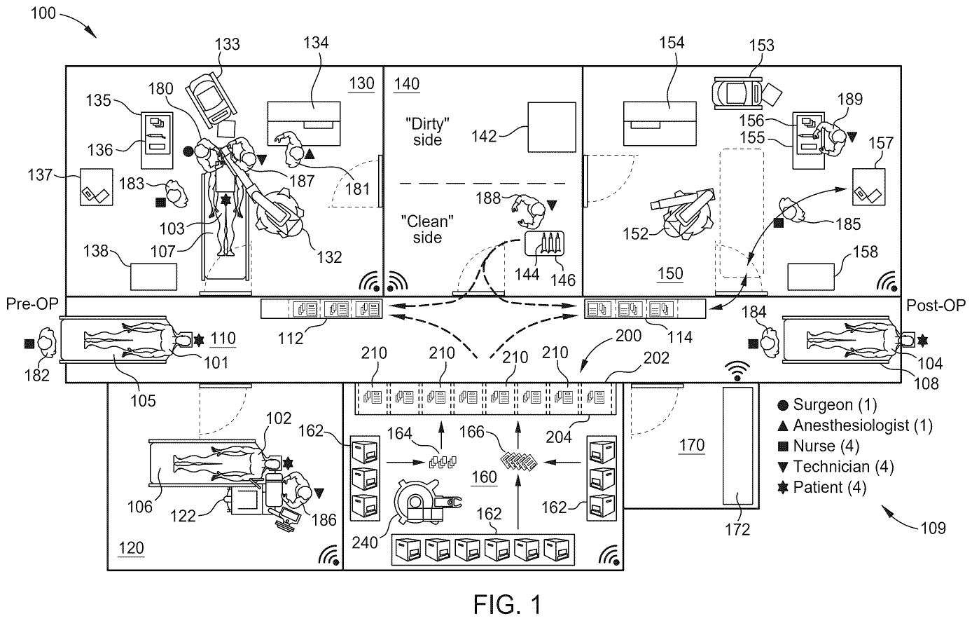

FIG. 1 depicts a schematic diagram of a surgical facility 100 for ophthalmic procedures, in accordance with embodiments of the present disclosure.

In certain embodiments, the surgical facility 100 may include a pre-operation (pre-op) room or holding area, a post-operation (post-op) room or holding area, a hallway 110, an eye imaging room 120, a first operating room 130, a sterilization room 140, a second operating room 150, a storage room 160, and a computer room 170. The rooms of the surgical facility 100 may be arranged around the hallway 110. The pre-op room and the post-op room may be located at opposite ends of the hallway 110. The first operating room 130, the sterilization room 140, and the second operating room 150 may be located on one side of the hallway 110, and the eye imaging room 120, the storage room 160, and the computer room 170 may be located on the other side of the hallway 110. Other arrangements, rooms, etc., are also supported, such as a single operating room 130, a hallway 110 that is located along one side of the surgical facility 100, etc.

The hallway 110 may include a first staging table 112 located proximate to the door of the first operating room 130, and a second staging table 114 located proximate to the door of the second operating room 150. In certain embodiments, each staging table 112, 114 may include an upper surface that may accommodate packages for four (4) ophthalmic procedures (as depicted in FIG. 1), which may be replenished by the surgical staff during the day. In other embodiments, each staging table 112, 114 may include an upper surface that may accommodate packages for all of the ophthalmic procedures that are scheduled for the first and second operating rooms 130, 150 (respectively). In some embodiments, each staging table 112, 114 may include an upper surface and one or more shelves that may accommodate packages for all of the ophthalmic procedures that are scheduled for the first and second operating rooms 130, 150 (respectively).

The eye imaging room 120 may include, inter alia, an imaging system 122, such as Alcon's LenSx system, etc. The sterilization room 140 may include, inter alia, an autoclave 142 (or other sterilization device) and a storage table 146 for reusable instruments 144 that have been sterilized and placed into sterile packages. The computer room 170 may include, inter alia, one or more control computers 172 that are configured to create and communicate with the storage, delivery, and setup robots over wireless communication links, such as WiFi, Bluetooth, etc.

The first operating room 130 may include, inter alia, a surgical microscope 132 mounted to an adjustable support, a surgical console 133 with a visualization system and a phaco handpiece (such as Alcon's Centurion® Vision System with Active Sentry® handpiece), an anesthesia console 134, a sterile table 135 configured to support a sterile tray 136 with the items that will be used during the ophthalmic procedure, a storage table 137, and a disposal station 138.

The second operating room 150 may be configured with the same equipment as the first operating room 130, including, inter alia, a surgical microscope 152 mounted to an adjustable support, a surgical console 153 with a visualization system and a phaco handpiece, an anesthesia console 154, a sterile table 155 configured to support a sterile tray 156 with the items that will be used during the ophthalmic procedure, a storage table 157, and a disposal station 158. Due to the location of the sterilization room 140 between the first operating room 130 and the second operating room 150, the layout of the second operating room 150 may be a mirror image of the layout of the first operating room 130.

The storage room 160 may include, inter alia, a dispenser 200, storage racks 162, and a storage robot (such as the storage robot 240). The dispenser 200 includes a front side 202, a back side 204, and compartments 210 that are accessible from the front side 202 and the back side 204. The storage robot is configured to stock the compartments 210 of the dispenser 200 with IOL packages 164 and surgical packages 166 from the storage racks 162. Advantageously, each compartment 210 stores the IOL packages 164 and the surgical packages 166 that are needed for a single ophthalmic procedure.

In the embodiment depicted in FIG. 1, before the ophthalmic procedures begin each day, the surgical staff may retrieve the packages that will be used during the day from the compartments 210 of the dispenser 200 and from the sterilization room 140, and stage the packages for each ophthalmic procedure on the staging tables 112, 114 outside the first and second operating rooms 130, 150. If the staging tables 112, 114 can not accommodate all of the packages that will be used during the day, then the surgical staff may replenish the staging tables 112, 114 during the day.

Four patients and ten surgical facility personnel are depicted in FIG. 1, which includes a legend 109.

The patient 101 is disposed on gurney 105, and is waiting in the pre-op area or just inside the hallway 110 until the eye imaging room 120 is available. Nurse 182 is attending patient 101.

The patient 102 is disposed on gurney 106 in the eye imaging room 120. The scrub technician 186 is located in the eye imaging room 120, and is performing the high-resolution imaging and measurement procedure on the patient 102 using the imaging system 122.

The patient 103 is disposed on gurney 107 in the first operating room 130. The surgeon 180, the anesthesiologist 181, the nurse 183, and the scrub technician 187 are located in the first operating room 130. The surgeon 180 is performing an ophthalmic procedure on an eye of the patient 103, assisted by the anesthesiologist 181, the nurse 183, and the scrub technician 187.

The scrub technician 188 is located in the sterilization room 140, and is sterilizing the reusable instruments from the first operating room 130 and the second operating room 150 using the autoclave 142 as needed.

The nurse 185 and the scrub technician 188 are located in the second operating room 150. The nurse 185 has already retrieved the surgical packages and instruments for the next ophthalmic procedure from the second staging table 114, deposited them on the storage table 157, and opened the IOL and surgical packages and the sterile package for the instrument. The scrub technician 188 is retrieving the items and the instrument from the open packages on the storage table 157, and arranging them on the sterile tray 156 on the sterile table 155.

The patient 104 is disposed on gurney 108, and is moving to the post-op area through hallway 110. Nurse 184 is attending patient 104.

When the ophthalmic procedure is completed, the surgeon 180 and the anesthesiologist 181 will move to the second operating room 150, and the nurse 185 will transport the patient 103 to the second operating room 150. The nurse 183 and the scrub technician 187 will then prepare the first operating room 130 for the next ophthalmic procedure on the patient 102. For example, nurse 183 and the scrub technician 187 will discard the detritus from the ophthalmic procedure into the disposal station 138, and provide any reusable instruments to the scrub technician 188 for sterilization in the sterilization room 140. The nurse 185 will retrieve the surgical packages and instruments for the next ophthalmic procedure from the first staging table 112, deposit them on the storage table 137, and open the IOL and surgical packages and the sterile package for the instrument. The scrub technician 187 will retrieve the items and the instrument from the open packages on the storage table 137, and arrange them on the sterile tray 136 on the sterile table 135.

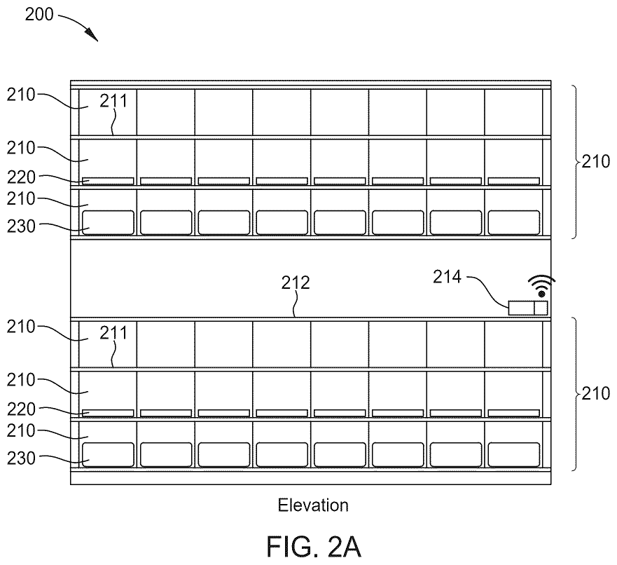

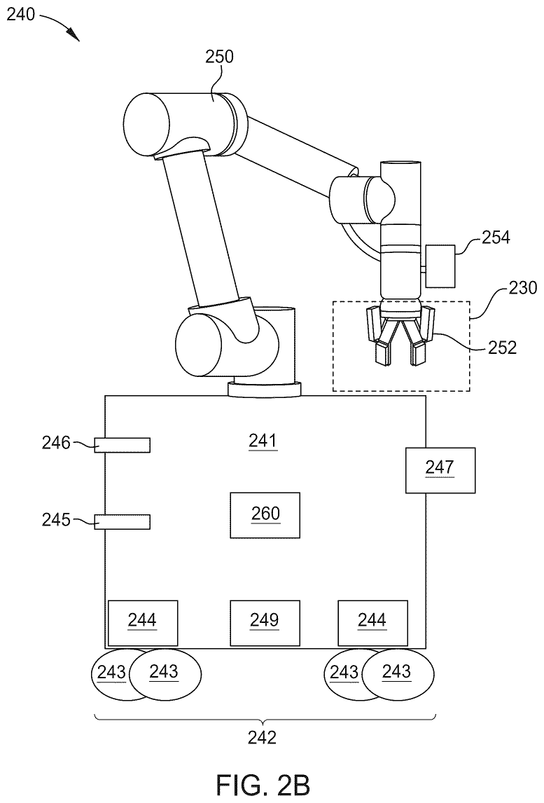

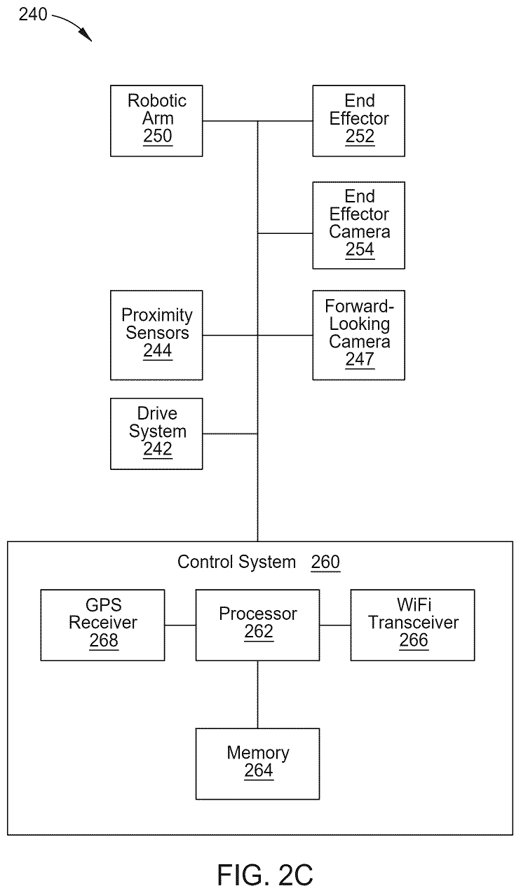

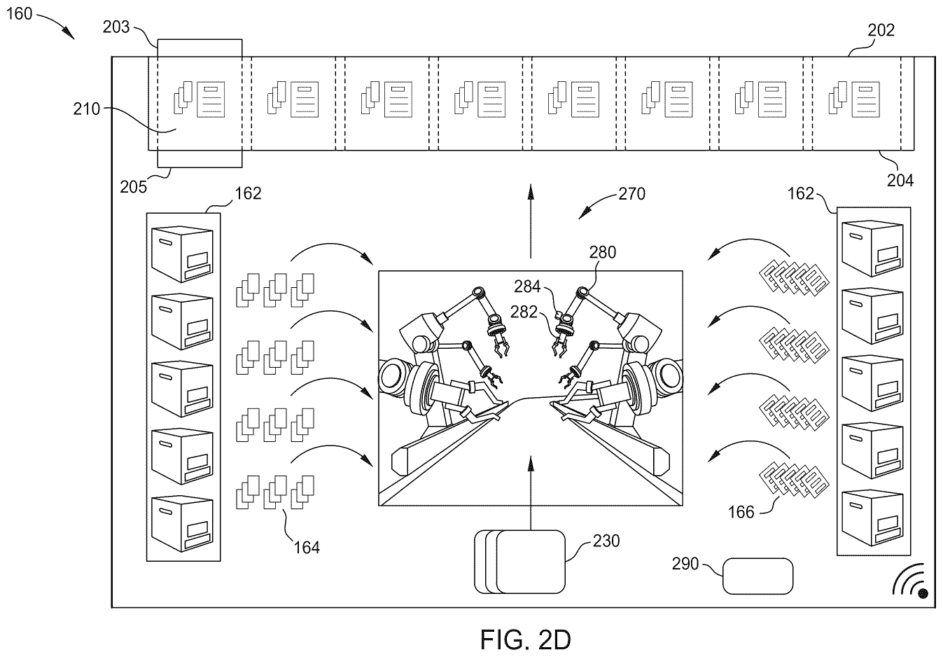

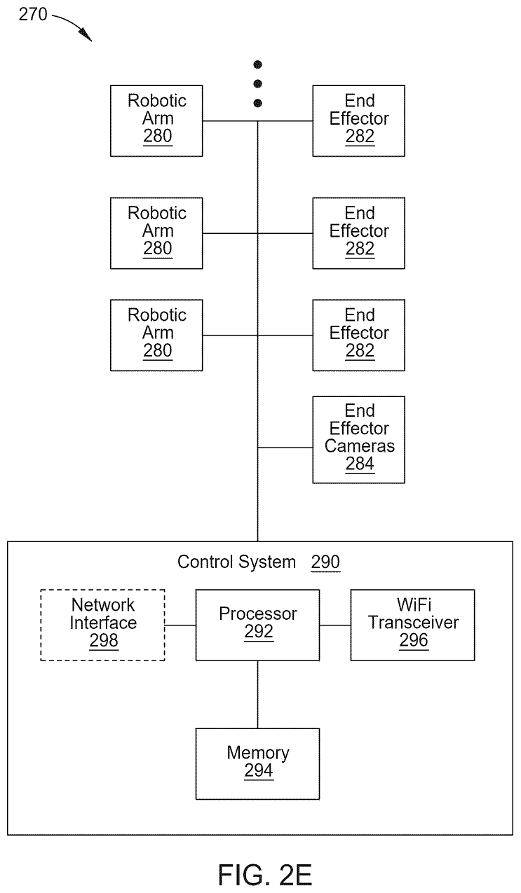

FIG. 2A depicts an elevation view of a dispenser 200 for the surgical facility 100, in accordance with embodiments of the present disclosure.

In certain embodiments, the dispenser 200 may include compartments 210 that are accessible from the front side 202 and the back side 204. In the embodiment depicted in FIG. 2A, the dispenser 200 includes three rows of eight compartments 210 that are arranged above a central shelf 212, and three rows of eight compartments 210 that are arranged below the central shelf 212. In one example, the surgeon 180 may perform an ophthalmic procedure about every 20 minutes during the day, which may be provisioned by about 24 compartments 210. Other arrangements and number of compartments 210 are also supported, such as three rows of eight compartments 210, six rows of four compartments 210, etc.

In certain embodiments, the compartments 210 may include openings on the front side 202 and the back side 204 (as depicted in FIG. 2A). In other embodiments, each compartment 210 may include a front door 203 (FIG. 2D) on the front side 202 and an opening on the back side 204. In some embodiments, each compartment 210 may include a front door 203 on the front side 202 and a back door 205 (FIG. 2D) on the back side 204. The doors may be manually operated, or remotely operated in response to a control signal.

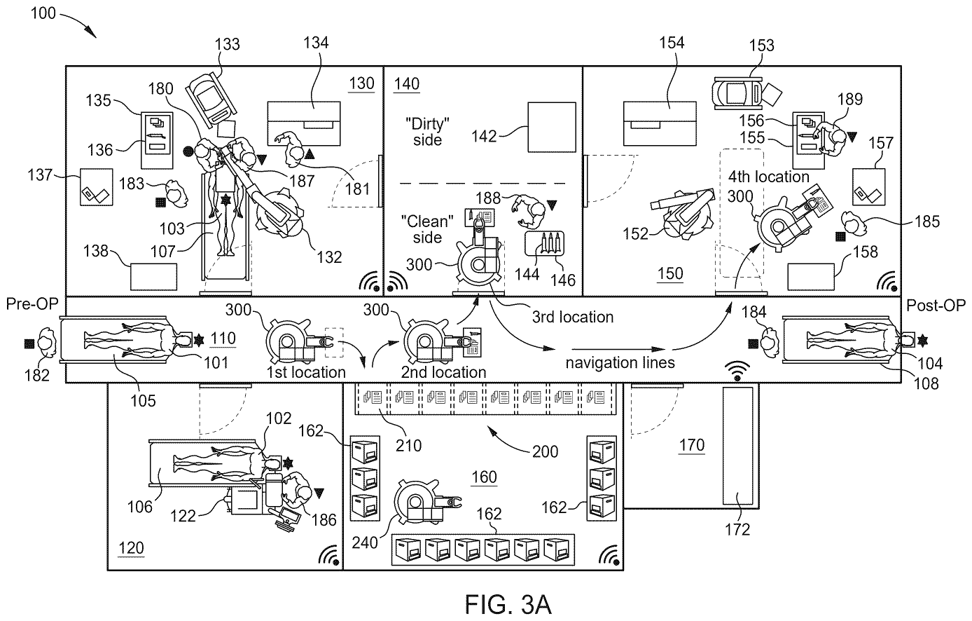

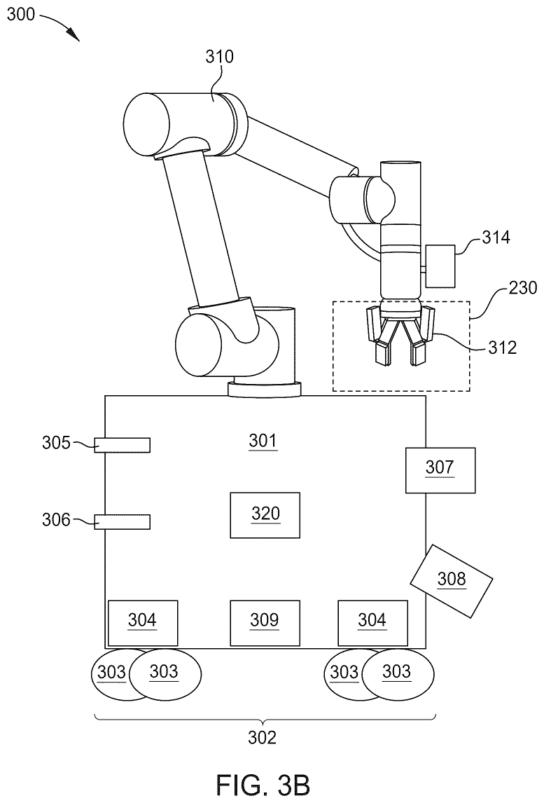

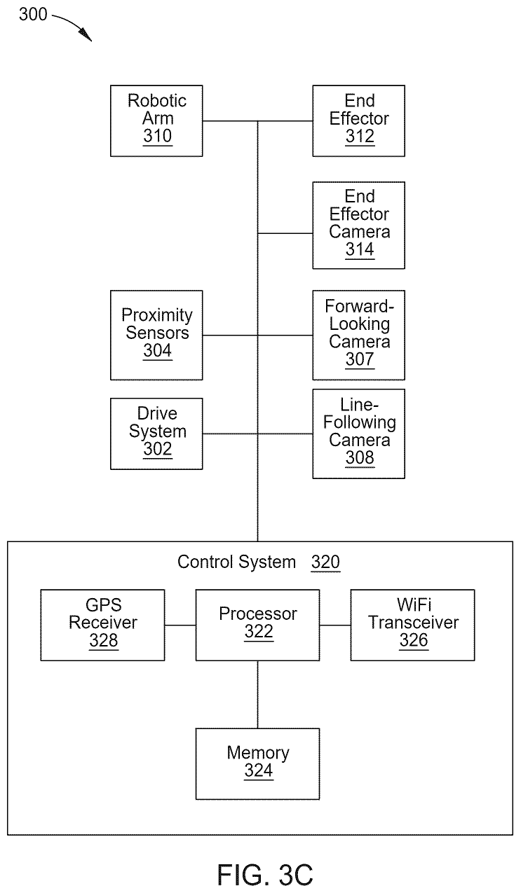

For example, each door may include a handle, a hinge, and a latch (such as a magnetic latch, a ball detent latch, etc.), and a nurse, a scrub technician, or an end effector of a robotic arm may grip the handle to open and close the door. In another example, the dispenser 200 may include a controller 214 with a wireless transmitter/receiver (or transceiver), and each door may include a motorized hinge that is coupled to the controller 214. The controller 214 may send a control signal to the motorized hinge to open and close the door in response to a command that is received by the wireless transceiver over a wireless network (such as WiFi, etc.). The command may be sent by the control computer 172, the storage robot 240, or the delivery robot 300 (FIG. 3B). In some embodiments, the control computer 172 may be coupled to the controller 214 over a wired network (such as Ethernet, etc.), and the command may be sent by the control computer 172 over the wired network.

The IOL packages 164 and the surgical packages 166 may be stored in several different ways in the compartments 210 of the dispenser 200. For example, the IOL packages 164 and the surgical packages 166 for an ophthalmic procedure may be stored on the floor 211 of a compartment 210. In another example, the IOL packages 164 and the surgical packages 166 for an ophthalmic procedure may be stored on a removable tray 220 in a compartment 210. In a further example, the IOL packages 164 and the surgical packages 166 for an ophthalmic procedure may be stored in a removable bin 230 in a compartment 210.

In certain embodiments, the IOL packages 164 and the surgical packages 166 for the ophthalmic procedures for the day may be stored in removable bins 230 in the compartments 210.

FIG. 2B depicts a storage robot 240, in accordance with embodiments of the present disclosure.

In certain embodiments, the storage robot 240 may include, inter alia, a chassis 241, a drive system 242 including articulated wheels 243, one or more proximity sensors 244, a WiFi antenna 245, a global positioning system (GPS) antenna 246, a forward-looking camera 247, a battery 249, a robotic arm 250 including an end effector 252 and an end effector camera 254, and a control system 260. In some embodiments, the chassis 241 may include an upper surface that is configured to receive a removable bin 230.

In certain embodiments, the drive system 242 may include four (4) articulated wheels 243 (as depicted in FIG. 2B), and each articulated wheel 243 may be coupled to a drive motor. The control system 260 may drive the articulated wheels 243 at different speeds in order to steer, direct, navigate, etc. the storage robot 240 to different locations within the storage room 160 (also known as differential steering). Additionally, each articulated wheel 243 may be independently steered by the control system 260. For example, the control system 260 may steer the storage robot 240 according to a particular steering technique, such as Ackermann steering, active-front-and-rear steering, spinning, crab steering, etc.

In other embodiments, at least one pair of articulated wheels 243 (such as the front pair or the back pair) may be coupled to a steering mechanism, and the control system 260 may navigate the storage robot 240 using the steering mechanism. In some embodiments, the front pair of articulated wheels 243 may be coupled to a steering mechanism, and the rear pair of articulated wheels 243 may be coupled to a drive motor. Other configurations of the drive system 242 are also supported, such as two (2) articulated wheels 243 with front and rear casters to provide balancing support for the chassis 241, etc.

The proximity sensors 244 may detect objects, such as the storage racks 162, the dispenser 200, the walls of the storage room 160, etc., that are close to the chassis 241 of the storage robot 240 without making physical contact with those objects. The proximity sensors 244 generate proximity sensor data (signals, etc.) that are provided to the control system 260. The proximity sensor data may include measurements of the distance between the proximity sensor 244 and the object. The proximity sensors 244 may include ultrasonic sensors, infrared transceivers, photoresistors, LED sensors, light detection and ranging (LIDAR) sensors, etc. In certain embodiments, the storage robot 240 may include a number of proximity sensors 244 that are distributed around the chassis 241 to provide up to 360° of detection coverage. For example, four proximity sensors 244 may each provide a 90° field-of-view, three proximity sensors 244 may each provide a 120° field-of-view, two proximity sensors 244 may each provide a 180° field-of-view, etc. In other embodiments, rather than providing 360° of coverage, the proximity sensors 244 may be distributed around the sides and rear of the chassis 241 to augment the image data provided by the forward-looking camera 247.

The forward-looking camera 247 is mounted to the front of the chassis 241, and generates image data that are provided to the control system 260 for various purposes, such as navigation, object detection, object recognition, etc. In certain embodiments, the forward-looking camera 247 may capture higher-quality images at a high resolution (such as 10 megapixels (MP), 13 MP, etc.) and a sharp focus. In some embodiments, the forward-looking camera 247 may also generate time-of-flight (ToF) data to capture a 3D map of the field-of-view, and the image data may include both red-green-blue (RGB) and depth video streams. In some embodiments, the forward-looking camera 247 may be a stereo camera that generates stereo image data that include stereo depth information. In some embodiments, the forward-looking camera 247 may be an infrared camera with an infrared light source (such as an LED) that generates image data without the need to illuminate the storage room 160 with artificial lighting (thereby providing a cost savings to the facility).

The robotic arm 250 may be a serial robotic arm, and may have 4 to 8 (or more) degrees of freedom. The degrees of freedom may be sufficient to position the end effector 252 of the robotic arm 250 at various three-dimensional positions and orientations within the working envelope of the robotic arm 250. The degrees of freedom may include one (or more) degrees of freedom of the end effector 252.

The end effector 252 may be a gripper a or similar end effector. The end effector 252 may also be a combination of a gripper and a sensor, etc. A gripper grabs and manipulates objects. A sensor provides measured data associated with an object or the surrounding environment, such as ultrasonic data, proximity data, force data, torque data, light data, etc.

In certain embodiments, the end effector 252 may be directly attached to the end or wrist of the robotic arm 250 using fasteners, such as screws, bolts, etc. In some embodiments, the end effector 252 may be directly attached to a mechanical coupling (such as a bracket, etc.) using fasteners, and the mechanical coupling may be directly attached to the end of the robotic arm 250 using fasteners. Alternatively, the mechanical coupling may be attached to an adapter plate that is directly attached to the end of the robotic arm 250.

In certain embodiments, the end or wrist of the robotic arm 250 may include a releasable coupling, such as a universal joint, a universal coupling, a quick change coupling, etc., that is configured to receive and secure different types of end effectors 252 to the robotic arm 250. For example, the releasable coupling may cooperate with a robotic arm mount located on the end effector 252 to secure the end effector 252 to the robotic arm 250.

In the embodiment depicted in FIG. 2B, the end effector 252 is a gripper that is configured to hold, manipulate, and release objects (such as an IOL package, a surgical package, a bin, etc.). The objects may have structures or features specifically designed to cooperate with the gripper. The gripper may be a mechanical gripper (such as a parallel-jaw gripper, three-finger gripper, a needle gripper, etc.), a magnetic or electromagnetic gripper, a pneumatic gripper, a vacuum gripper, an electric gripper, a hydraulic gripper, etc.

Precise positioning of the end effector 252 may be performed in various ways. In certain embodiments, a kinematic state of the storage robot 240 and a known mapping of the objects in the storage room 160 may be used along with obstacle detectors, such as the proximity sensors 244, the forward-looking camera 247, etc., to position the end effector 252. In other embodiments, one (or more) end effector cameras 254 may mounted to the robotic arm 250 on or near the end effector 252. For example, an end effector camera 254 may be mounted within 15 cm of, and rigidly coupled to, the end effector 252. Images from the end effector camera 254 may be processed to determine the location and orientation of the end effector 252, and used as feedback to the control system 260 to control the robotic arm 250. The location and orientation data may be used to control the robotic arm 250 to achieve a desired position and orientation of the end effector 252.

FIG. 2C depicts a block diagram of the storage robot 240 depicted in FIG. 2B, in accordance with embodiments of the present disclosure.

The control system 260 is coupled to the drive system 242, the proximity sensors 244, the forward-looking camera 247, the robotic arm 250, the end effector 252, and the end effector camera 254. In certain embodiments, the control system 260 may include a processor 262 coupled to a memory 264, a WiFi transceiver 266, and a GPS receiver 268. The WiFi transceiver 266 is coupled to the WiFi antenna 245, and the GPS receiver 268 is coupled to the GPS antenna 246. The WiFi antenna 245 and the GPS antenna 246 may be mounted to an external surface of the chassis 241 to improve signal transmission and reception. In some embodiments, the WiFi transceiver 266 and the GPS receiver 268 may incorporate antennae within their respective form factors.

Generally, the storage robot 240 may fill, stock, replenish, etc., the compartments 210 of the dispenser 200 with certain IOL and surgical packages will be needed for the ophthalmic procedures that are scheduled for the first and second operating rooms 130, 150 for the next day. Advantageously, the storage robot 240 may fill the compartments 210 for the next day's ophthalmic procedures when the surgical facility 100 is closed for the day, such as between 8 pm and 6 am, and may not require the use of artificial lighting in the storage room 160. Alternatively, the storage robot 240 may fill the compartments 210 for the day's ophthalmic procedures on the same day.

Certain ophthalmic procedures may require one or more IOL packages 164 and one or more surgical packages 166 that are stored in the storage racks 162. For example, a patient undergoing a ophthalmic procedure on both eyes may require an IOL package 164 for each eye. In another example, a patient undergoing a ophthalmic procedure on one eye may only require an IOL package 164 for that eye. In a further example, a single IOL package 164 may be sufficient for a patient undergoing a ophthalmic procedure on both eyes. Certain ophthalmic procedures may not require an IOL package 164 at all, and may only require one or more surgical packages 166.

In certain embodiments, the control system 260 may navigate to a storage rack 162 within the storage room 160 based on sensor data, retrieve a surgical package 166 (or an IOL package 164) from a storage rack 162 using the robotic arm 250 and the end effector 252, navigate to the back side 204 of the dispenser 200 based on the sensor data, and deliver the surgical package 166 (or the IOL package 164) to a compartment 210 of the dispenser 200 using the robotic arm 250 and the end effector 252. The sensor data may include proximity data from the proximity sensors 244, image data from the forward-looking camera 247, and position data from the GPS receiver 268.

A map of the storage room 160 may be stored in the memory 264 of the control system 260, which may include the location and dimensions of the storage racks 162, the back side 204 of the dispenser 200, and the walls of the storage room 160, as well as any obstacles in the storage room 160. The control system 260 (such as the processor 262) may process the sensor data and execute one or more models (such as machine learning models, etc.), etc., to determine a current location based on the sensor data and the map, determine a desired location based on the map, navigate to the desired location based on the sensor data and the map, and then perform the appropriate function.

For example, the storage robot 240 may be located adjacent to a storage rack 162 (the current location) after retrieving a surgical package 166 from the storage rack 162 using the robotic arm 250 and the end effector 252. The desired location may be adjacent to a particular compartment 210 in the dispenser 200, and the control system 260 may then navigate from the storage rack 162 to the back side 204 of the dispenser 200. The control system 260 may then place the surgical package 166 into a compartment 210 using the robotic arm 250 and the end effector 252.

In certain embodiments, the control computer 172 may transmit a “dispenser” command to the storage robot 240 over a wireless communication link. The dispenser command may include a compartment list that identifies the compartments 210 that are to be used to store the packages for the ophthalmic procedures that are scheduled for the next day, such as the IOL packages 164, the surgical packages 166, etc. The dispenser command may also include an item list for each compartment 210 that identifies the packages that will be stored in that compartment 210. In response to receiving the dispenser command, the control system 260 begins to fill each compartment 210 in the compartment list with the packages in each item list. When the last compartment 210 in the compartment list has been filled, the control system 260 may transmit data, such as a “dispenser” response, etc., to the control computer 172 that indicates that the dispenser 200 is ready for the ophthalmic procedures that are scheduled for the next day (or that day).

In other embodiments, the control computer 172 may transmit a “compartment” command to the storage robot 240 over a wireless communication link. The compartment command may be directed to a single compartment 210, and may include an item list that identifies the packages that will be stored in that compartment 210. In response to receiving the compartment command, the control system 260 begins to fill that compartment 210 with the packages in the item list. When that compartment 210 has been filled, the control system 260 may transmit data, such as a compartment response, etc., to the control computer 172 that indicates the compartment 210 has been filled. In response, the control computer 172 may transmit another compartment command to the storage robot 240 over the wireless communication link that is directed to the next compartment, and so on.

As discussed above, the IOL packages 164 and the surgical packages 166 may be stored on the floor 211 of each compartment 210, on a removable tray 220 in each compartment 210, or in a removable bin 230 in each compartment 210.

In certain embodiments, the control system 260 may navigate to the back side 204 of the dispenser 200 based on the sensor data, retrieve a removable bin 230 from a particular compartment 210 of the dispenser 200 using the robotic arm 250 and the end effector 252, and place the removable bin 230 on the upper surface of the chassis 241. The control system 260 may then fill the removable bin 230 with all of the IOL packages 164 and the surgical packages 166 that are needed for a particular ophthalmic procedure, navigate to the back side 204 of the dispenser 200, and then place the removable bin 230 into the same compartment 210.

More particularly, the control system 260 may navigate to a storage rack 162 within the storage room 160 based on sensor data, retrieve a first surgical package 166 from a storage rack 162 using the robotic arm 250 and the end effector 252, and deposit the first surgical package 166 into the removable bin 230. The control system 260 may navigate to another storage rack 162 within the storage room 160 based on sensor data, retrieve a second surgical package 166 from a storage rack 162 using the robotic arm 250 and the end effector 252, and deposit the second surgical package 166 into the removable bin 230. The control system 260 may navigate to another storage rack 162 within the storage room 160 based on sensor data, retrieve a first IOL package 164 from a storage rack 162 using the robotic arm 250 and the end effector 252, and deposit the first IOL package 164 into the removable bin 230. And so on.

After all of the IOL packages 164 and the surgical packages 166 that are needed for the particular ophthalmic procedure have been deposited into the removable bin 230, the control system 260 may navigate to the back side 204 of the dispenser 200 based on the sensor data, and deliver the removable bin 230 to a particular compartment 210 of the dispenser 200 using the robotic arm 250 and the end effector 252. And so on.

FIG. 2D depicts a schematic diagram of the storage room 160 with another storage robot 270, in accordance with embodiments of the present disclosure.

In certain embodiments, the storage robot 270 may include, inter alia, a conveyor 272, a number of robotic arms 280, and a control system 290. The conveyor 272 may be a belt conveyor system with a powered or unpowered conveyer belt, a roller conveyor system with powered or unpowered rollers, etc. The robotic arms 280 may be located along either side of the conveyor 272, a supply of bins 230 may be located near a front end of the conveyor 272, and the dispenser 200 may be located near a back end of the conveyor 272. The conveyor 272 is configured to transport bins 230 from the front end to the back end. For a powered conveyor system, the conveyor 272 may include motors and sensors that are coupled to the control system 290 to control the movement of the bins 230. For an unpowered conveyor system, the robotic arms 280 may push the bins 230 along the conveyor 272, grip and translate the bins 230 along the conveyor 272, etc.

Each robotic arm 280 includes an end effector 282 and an end effector camera 284, and has an area of operation or working envelope that encompasses a portion of the conveyor 272 and one or more storage racks 162. Additionally, the robotic arms 280 at the front end of the conveyor 272 may have working envelopes that encompass the supply of bins 230, while the robotic arms 280 at the back end of the conveyor 272 may have working envelopes that encompass at least a portion of the back side 204 of the dispenser 200.

In certain embodiments, the storage robot 270 may include six (6) robotic arms 280 (as depicted in FIG. 2D). Three (3) robotic arms 280 may be arranged on one side of the conveyor 272 (such as the left side), and three (3) robotic arms 280 may be arranged on the other side of the conveyor 272 (such as the right side). Other numbers and arrangements of robotic arms 280 are also supported. For example, one robotic arm 280 may be located at the front end of the conveyor 272, one robotic arm 280 may be located at the back end of the conveyor 272, and two (4) robotic arms 280 may be arranged on each side of the conveyor 272. In another example, the robotic arms 280 may be arranged on the same side of the conveyor 272 (such as the left side or the right side), such as two (2) robotic arms 280, there (3) robotic arms 280, four (4) robotic arms 280, etc.

Generally, each robotic arm 280 may be similar to the robotic arm 250 of the storage robot 240. For example, each robotic arm 280 may be a serial robotic arm, and may have 4 to 8 (or more) degrees of freedom. The degrees of freedom may be sufficient to position the end effector 282 of the robotic arm 280 at various three-dimensional positions and orientations within the working envelope of the robotic arm 280. The degrees of freedom may include one (or more) degrees of freedom of the end effector 282.

Each end effector 282 may be a gripper or similar end effector, as described above. Each end effector 282 may also be a combination of a gripper and a sensor, etc. In the embodiment depicted in FIG. 2D, the end effector 282 is a gripper for holding and releasing objects, such as IOL packages 164, surgical packages 166, bins 230, etc.

Precise positioning of the end effectors 282 of the robotic arms 280 may be performed in various ways. In certain embodiments, a kinematic state of the storage robot 270 and a known mapping of the objects in the storage room 160 may be used along with obstacle sensors, such as proximity sensors, cameras disposed around the storage room 160, etc., to position the end effectors 282. In other embodiments, the end effector cameras 284 may be mounted to the robotic arms 280 on or near the end effectors 282. For example, each end effector camera 284 may be mounted within 15 cm of, and rigidly coupled to, the respective end effector 282. Images from the end effector cameras 284 may be processed to determine the location and orientation of the end effectors 282, and used as feedback to the control system 290 to control the robotic arms 280. The location and orientation data may be used to control the robotic arms 280 to achieve a desired position and orientation of the end effectors 282.

FIG. 2E depicts a block diagram of the storage robot 270 depicted in FIG. 2D, in accordance with embodiments of the present disclosure.

The control system 290 is coupled to the conveyor 272, the robotic arms 280, the end effectors 282, and the end effector cameras 284. In certain embodiments, the control system 290 may include a processor 292 coupled to a memory 294 and a WiFi transceiver 296. In some embodiments, the control system 290 may include a network interface 298 that is coupled to the processor 292, and coupled to the control computer 172 over a wired network (such as Ethernet, etc.). The WiFi transceiver 266 may be coupled to an external WiFi antenna, or the WiFi transceiver 266 may incorporate an antenna within its form factor.

Generally, the storage robot 270 may fill, stock, replenish, etc., the bins 230 stored within the compartments 210 of the dispenser 200 with certain packages will be needed for the ophthalmic procedures that are scheduled for the first and second operating rooms 130, 150 for the next day (or that day). Advantageously, the storage robot 270 may fill the bins 230 within the compartments 210 for the next day's ophthalmic procedures when the surgical facility 100 is closed for the day, such as between 8 pm and 6 am, and may not require the use of artificial lighting in the storage room 160. Alternatively, the storage robot 270 may fill the bins 230 within the compartments 210 for the day's ophthalmic procedures on the same day.

A map of the storage room 160 may be stored in the memory 294 of the control system 290, which may include the location and dimensions of the storage racks 162, the back side 204 of the dispenser 200, and the walls of the storage room 160, as well as any obstacles in the storage room 160. The control system 290 (such as the processor 292) may process image data generated by the end effector cameras 284, and execute one or more models (such as machine learning models, etc.), etc., to determine a current position and orientation for each robotic arm 280 and end effector 282 based on the image data and the map, determine a desired position and orientation for each robotic arm 280 and end effector 282 based on the map, and then move the robotic arms 280 and end effectors 282 to the desired positions and orientations.

In certain embodiments, the control computer 172 may transmit the dispenser command to the storage robot 270 over a wireless (or wired) communication link. The dispenser command may include a compartment list that identifies the compartments 210 that are to be used to store the packages for the ophthalmic procedures that are scheduled for the next day, such as the IOL packages 164, the surgical packages 166, etc. The dispenser command may also include an item list for each compartment 210 that identifies the packages that will be stored in that compartment 210. In response to receiving the dispenser command, the control system 290 fills the bin 230 for the first compartment 210 in the compartment list with the packages in each item list, and then deposits the bin 230 in the first compartment 210. The bins 230 for remaining compartment 210 in the compartment list are filled in sequence, and when the bin 230 for the last compartment 210 in the compartment list has been filled, the control system 290 may transmit data, such as a “dispenser” response, etc., to the control computer 172 that indicates that the dispenser 200 is ready for the ophthalmic procedures that are scheduled for the next day (or that day).

In certain embodiments, the control computer 172 may transmit the compartment command to the storage robot 240 over a wireless (or wired) communication link. The compartment command may be directed to a single compartment 210, and may include an item list that identifies the packages to be stored in that compartment 210. In response to receiving the compartment command, the control system 260 fills the bin 230 for the compartment 210 with the packages in the item list, deposits the bin 230 in the compartment 210, and then transmits data, such as a compartment response, etc., to the control computer 172 that indicates the compartment 210 has been filled. In response, the control computer 172 may transmit another compartment command to the storage robot 270 over the wireless communication link that is directed to the next compartment, and so on.

The following description of the process to fill a bin 230 for a compartment 210 employs an embodiment that includes an arrangement of three (3) robotic arms 280 located on one side of the conveyor 272 (such as the left side): a first robotic arm 280 that is located near the front of the conveyor 272, a second robotic arm 280 that is located near the center of the conveyor 272, and a third second robotic arm 280 that is located near the back end of the conveyor 272.

A compartment command to fill a bin 230 for a particular compartment 210 is received by the control system 290 over a wireless (or wired) communication link. The compartment command includes an item list with a first surgical package 166 stored in a first storage rack 162, a second surgical package 166 stored in a second storage rack 162, and an IOL package 164 stored in a third storage rack 162.

In response to receiving the compartment command, the control system 290 commands the first robotic arm 280 to retrieve an empty bin 230 from the supply of bins 230 using the end effector 282 (such as a gripper), and place the bin 230 on the front end of the conveyor 272. The control system 290 then commands the first robotic arm 280 to retrieve the first surgical package 166 from the first storage rack 162 using the end effector 282, and deposit the first surgical package 166 into the bin 230. The control system 290 then commands the conveyor 272 to advance the bin 230 to the center of the conveyor 272. The control system 290 then commands the second robotic arm 280 to retrieve the second surgical package 166 from the second storage rack 162 using the end effector 282, and deposit the second surgical package 166 into the bin 230. The control system 290 then commands the conveyor 272 to advance the bin 230 to the back end of the conveyor 272. The control system 290 then commands the third robotic arm 280 to retrieve the IOL package 164 from the third storage rack 162 using the end effector 282, and deposit the IOL package 164 into the bin 230. The control system 290 then commands the third robotic arm 280 to grip the bin 230 and deposit the bin 230 into the particular compartment 210.

Other arrangements of robotic arms 280 and sequences of IOL and surgical package retrieval are also supported. Generally, the control system 290 may command the robotic arm 280 that is closest to the particular storage rack 162 that contains the desired package to retrieve and place the package in the bin 230.

Advantageously, an inventory of the IOL and surgical packages placed in the bins 230 may be automatically tracked by the control computer 172 through the dispenser and compartment responses. Additionally, when an IOL or a surgical package is not present in a particular storage rack 162, the control system 290 may transmit data, such as an “error” response, etc., to the control computer 172. The error response may indicate that the IOL or surgical package was not present in the storage rack 162.

FIG. 3A depicts another schematic diagram of the surgical facility 100 for ophthalmic procedures, in accordance with embodiments of the present disclosure.

In certain embodiments, the surgical facility 100 may include a pre-operation (pre-op) room or holding area, a post-operation (post-op) room or holding area, a hallway 110, an eye imaging room 120, a first operating room 130, a sterilization room 140, a second operating room 150, a storage room 160, and a computer room 170, as described above with respect to FIG. 1. The surgical facility 100 also includes a delivery robot 300, and the staging tables 112, 114 in the hallway 110 are not needed.

The delivery robot 300 navigates between the hallway 110, the first operating room 130, the sterilization room 140, and the second operating room 150 in order to retrieve and deliver the packages that are needed for the ophthalmic procedures that are performed in the first operating room 130 and the second operating room 150. Rather than staging the packages that are needed for all of the ophthalmic procedures on the staging tables 112, 114 (see FIG. 1), the delivery robot 300 advantageously retrieves the packages needed for the next ophthalmic procedure from the dispenser 200 and the sterilization room 140 (when a reusable instrument is needed for the ophthalmic procedure), and delivers the packages to the storage table 137 in the first operating room 130 or to the storage table 157 in the second operating room 150.

The delivery robot 300 is depicted in four different locations during the process of retrieving and delivering the packages needed for the next ophthalmic procedure in the second operating room 150. The first and second locations are in the hallway 110, the third location is in the sterilization room 140, and the fourth location is in the second operating room 150. After the delivery robot 300 delivers the packages to the storage table 157 in the second operating room 150, the nurse 185 may open the IOL, surgical, and sterile packages. The scrub technician 187 may retrieve the items from the open packages on the storage table 137, and arrange them on the sterile tray 136 on the sterile table 135. Alternatively, the scrub technician 187 may arrange the items directly on the sterile table 135.

FIG. 3B depicts a diagram of a delivery robot 300, in accordance with embodiments of the present disclosure.

In certain embodiments, the delivery robot 300 may include, inter alia, a chassis 301, a drive system 302 including four (4) articulated wheels 303, one or more proximity sensors 304, a WiFi antenna 305, a GPS antenna 306, a forward-looking camera 307, a line-following camera 308, a battery 309, a robotic arm 310 including an end effector 312 (such as a gripper) and an end effector camera 314, and a control system 320. In some embodiments, the chassis 301 may include an upper surface that is configured to receive a removable bin 230.

Generally, the delivery robot 300 may be configured with the same components as the storage robot 240. In certain embodiments, the drive system 302 may be the same as the drive system 242, the proximity sensors 304 may be the same as the proximity sensors 244, the forward-looking camera 307 may be the same as the forward-looking camera 247, the robotic arm 310, end effector 312 and end effector camera 314 may be the same as the robotic arm 250, end effector 252 and end effector camera 254, etc. In other embodiments, the delivery robot 300 may be configured with different components that perform the same functions.

The line-following camera 308 is mounted to the front of the chassis 301 at a downward angle (such as 30°, 45°, 60°, etc.), and generates image data that are provided to the control system 320. The image data may be used for navigation as well as other purposes, such as object detection, etc. For example, the line-following camera 308 may detect navigation lines on the floor of the surgical facility 100 that delineate one or more paths or tracks that link the hallway 110 with the first operating room 130, the sterilization room 140, and the second operating room 150. The navigation lines may be detectable in the visible spectrum, the infrared spectrum, or both.

In certain embodiments, the line-following camera 308 may capture lower-quality images at a lower resolution (such as 2 MP, 4 MP, 6 MP, etc.) and a sharp focus. In some embodiments, the line-following camera 308 may be an infrared camera with an infrared light source (such as an LED).

FIG. 3C depicts a block diagram of the delivery robot 300 depicted in FIG. 3B, in accordance with embodiments of the present disclosure.

The control system 320 is coupled to the drive system 302, the proximity sensors 304, the forward-looking camera 307, the line-following camera 308, the robotic arm 310, the end effector 312, and the end effector camera 314. In certain embodiments, the control system 320 may include a processor 322 coupled to a memory 324, a WiFi transceiver 326, and a GPS receiver 328. The WiFi transceiver 326 is coupled to the WiFi antenna 305, and the GPS receiver 328 is coupled to the GPS antenna 306. The WiFi antenna 305 and the GPS antenna 306 may be mounted to an external surface of the chassis 301 to improve signal transmission and reception. In some embodiments, the WiFi transceiver 326 and the GPS receiver 328 may incorporate antennae within their respective form factors.

In certain embodiments, the control system 320 may navigate to the dispenser 200 based on sensor data, retrieve a bin 230 containing one or more surgical packages 166 from a compartment 210 in the dispenser 200 using the robotic arm 310 and the end effector 312, navigate to the first operating room 130 (or the second operating room 150) based on the sensor data, and deliver the contents of the bin 230 to the storage table 137 (or the storage table 157) using the robotic arm 310 and the end effector 312. The sensor data may include proximity data from the proximity sensors 304, image data from the forward-looking camera 307, image data from the line-following camera 308, and position data from the GPS receiver 328.

In certain embodiments, the control system 320 may navigate to the sterilization room 140 based on the sensor data, retrieve a reusable instrument 144 from the storage table 146 in the sterilization room 140, navigate to the first operating room 130 (or the second operating room 150) based on the sensor data, and deliver the IOL packages 164, the surgical packages 166, and the reusable instrument 144 to the storage table 137 (or the storage table 157) using the robotic arm 310 and the end effector 312.

A map of the hallway 110, the first operating room 130, the sterilization room 140, and the second operating room 150 may be stored in the memory 324 of the control system 320. The map may include, inter alia, the dimensions of the hallway 110, the first operating room 130, the sterilization room 140, and the second operating room 150, the navigation lines, and the location and dimensions of the obstacles in the first operating room 130, the sterilization room 140, and the second operating room 150, such as instruments, consoles, tables, stations, etc.

The control system 320 (such as the processor 322) may process the sensor data and execute one or more models (such as machine learning models, etc.), etc., to determine a current location based on the sensor data and the map, determine a desired location based on the map, navigate to the desired location based on the sensor data and the map, and then perform the appropriate function.

For example, the delivery robot 300 may be located in the hallway 110 adjacent to a particular compartment 210 in the dispenser 200 (the current location) after retrieving a bin 230 from the compartment 210 using the robotic arm 310 and the end effector 312. The bin 230 may be received by the upper surface of the chassis 301. The desired location may be adjacent to the storage table 146 in the sterilization room 140, and the control system 320 may then navigate from the dispenser 200 to the storage table 146. The control system 320 may then place a reusable instrument 144 from the storage table 146 into the bin 230 using the robotic arm 250 and the end effector 252.

In certain embodiments, the control computer 172 may transmit a “next procedure” command to the delivery robot 300 over a wireless communication link. The next procedure command may be directed to the first operating room 130 (or the second operating room 150), and a particular compartment 210 in the dispenser 200. The next procedure command may also include an item list that identifies the packages that are stored in the bin 230, and an instrument list that identifies the instruments that need to be retrieved from the sterilization room 140.

In response to receiving the next procedure command, the control system 320 retrieves the bin 230 from the compartment 210 in the dispenser 200, retrieves the instrument (if identified) from the sterilization room 140 and places the instrument in the bin 230, and delivers the contents of the bin 230 to the first operating room (or the second operating room 150). After the contents of the bin 230 have been delivered, the control system 320 may transmit data, such as a next procedure response, etc., to the control computer 172 that indicates that the contents of the bin 230 have been delivered. In response, the control computer 172 may transmit another next procedure command to the delivery robot 300 over the wireless communication link, and so on.

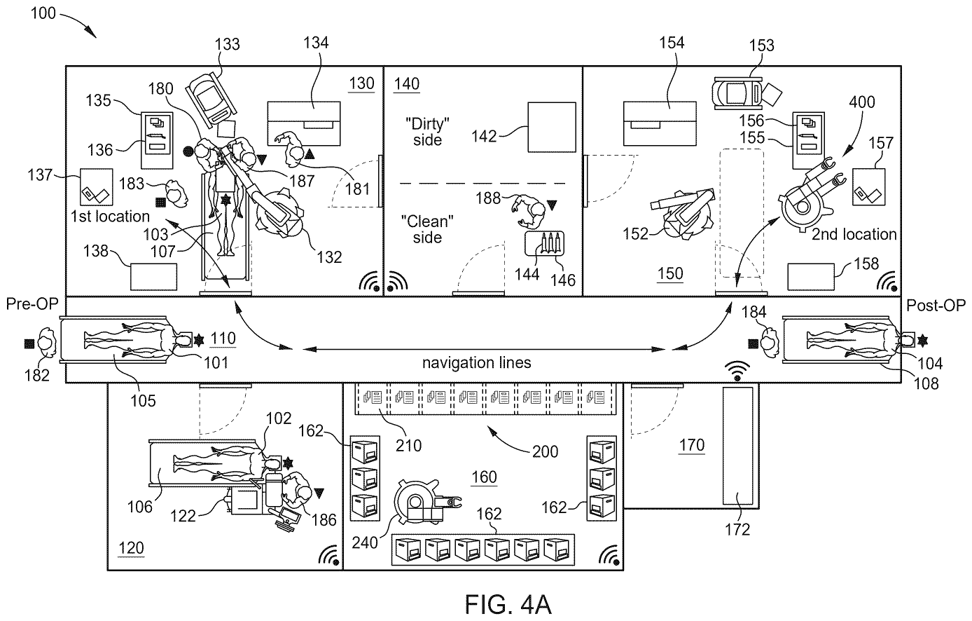

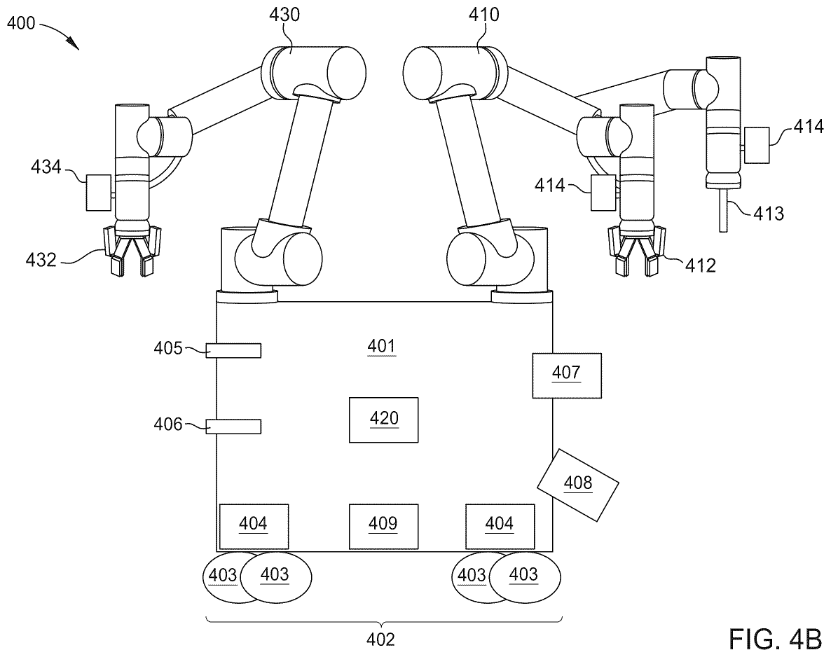

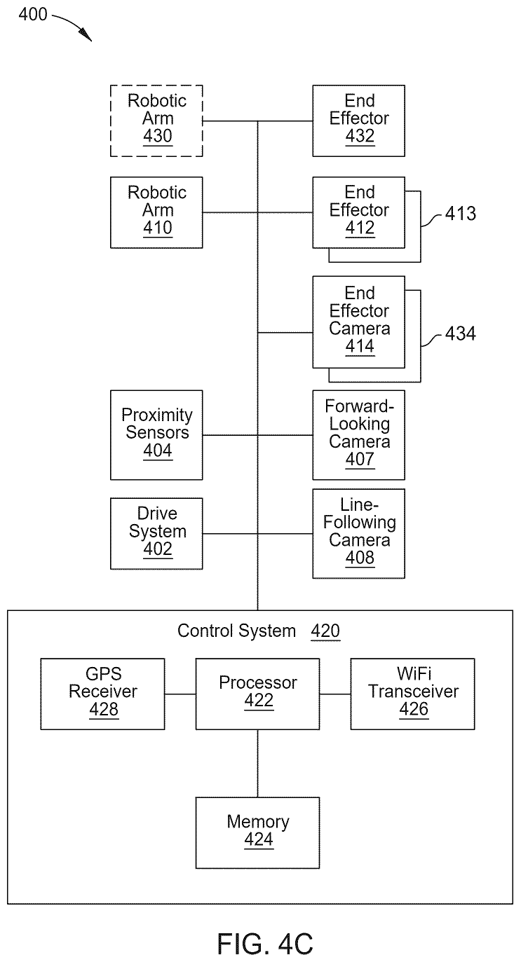

FIG. 4A depicts another schematic diagram of the surgical facility 100 for ophthalmic procedures, in accordance with embodiments of the present disclosure.

In certain embodiments, the surgical facility 100 may include a pre-operation (pre-op) room or holding area, a post-operation (post-op) room or holding area, a hallway 110, an eye imaging room 120, a first operating room 130, a sterilization room 140, a second operating room 150, a storage room 160, and a computer room 170, as described above with respect to FIG. 1. The surgical facility 100 also includes a setup robot 400.

The setup robot 400 navigates between the hallway 110, the first operating room 130, and the second operating room 150 in order to prepare the items that are needed for the ophthalmic procedures that are performed in the first operating room 130 and the second operating room 150. Advantageously, the setup robot 400 may reduce or eliminate the need for one nurse and one scrub technician, such as the nurse 185 and the scrub technician 189 stationed in the second operating room 150. The nurse 183 and the scrub technician 187 would then rotate between the first operating room 130 and the second operating room 150 with the surgeon 180 and the anesthesiologist 181.

The setup robot 400 is depicted in a second location that may be used during the process of preparing the items needed for the next ophthalmic procedure in the second operating room 150. The second location for the setup robot 400 is adjacent to the sterile table 155 and the storage table 157. The first location for the setup robot 400 is adjacent to the sterile table 135 and the storage table 137 in the first operating room 130, and may be used during the process of preparing the items needed for the next ophthalmic procedure in the first operating room 130.

As discussed above, the packages needed for the next ophthalmic procedure may include one or more IOL packages 164, one or more surgical packages 166, and a sterile package for a reusable instrument 144. As depicted in FIG. 4A, the packages needed for the next ophthalmic procedure in the second operating room 150 have already been delivered to the storage table 157. In certain embodiments, in response to receiving a next procedure command from the control computer 172, a delivery robot 300 may retrieve a bin 230 from a compartment 210 in the dispenser 200, retrieve a sterile package from the sterilization room 140 (if needed) and place it in the bin 230, and deliver the contents of the bin 230 to the storage table 157 (as depicted FIG. 3A). In other embodiments, the nurse 183 may retrieve the packages needed for the next ophthalmic procedure from the second staging table 114 (as depicted FIG. 1), and deposit them on the storage table 157.

After the IOL, surgical, and sterile packages are delivered to the storage table 157 in the second operating room 150, the setup robot 400 may open the packages, remove the items from the packages, and then deposit the items on the sterile tray 156 on the sterile table 155.

In certain embodiments, the setup robot 400 may cut open each package using a robotic arm with non-sterile end effectors, and then invert each package over the sterile tray 156 to remove the items and deposit them on the sterile tray 156. In other embodiments, the setup robot 400 may cut open each package using a first robotic arm with non-sterile end effectors, remove each item from the packages using a second robotic arm with a sterile end effector, and then arrange each item on the sterile tray 156 using the second robotic arm and the sterile end effector.

FIG. 4B depicts a diagram of a setup robot 400, in accordance with embodiments of the present disclosure.

In certain embodiments, the setup robot 400 may include, inter alia, a chassis 401, a drive system 402 including four (4) articulated wheels 403, one or more proximity sensors 404, a WiFi antenna 405, a GPS antenna 406, a forward-looking camera 407, a line-following camera 408, a battery 409, a robotic arm 410 including end effectors 412, 413 and end effector cameras 414, and a control system 420.

Generally, the setup robot 400 may be configured with the same components as the delivery robot 300. In certain embodiments, the drive system 402 may be the same as the drive system 302, the proximity sensors 404 may be the same as the proximity sensors 304, the forward-looking camera 407 may be the same as the forward-looking camera 307, the line-following camera 408 may be the same as the line-following camera 308, the robotic arm 410 and end effector camera 414 may be the same as the robotic arm 310 and end effector camera 314, etc. In other embodiments, the setup robot 400 may be configured with different components that perform the same functions.

The end effector 412 may be a gripper. Generally, the end effector 413 is configured to open the IOL, surgical, and sterile packages, and may be removable and autoclavable. In certain embodiments, the end effector 413 may be a cutting tool, such as a knife, a circular saw blade, etc., that is configured to cut through the material of the IOL, surgical, and sterile packages to create an opening in the package. In other embodiments, the end effector 413 may be a vacuum tool, a heat plate, etc. Commonly-owned U.S. patent application Ser. No. 63/681,603 (filed on August 9, 2024) describes different end effectors that may be used to manipulate and open surgical packages, and is incorporated herein by reference in its entirety.

In certain embodiments, the setup robot 400 may include a second robotic arm 430 including an end effector 432 and a camera 434. The end effector 432 may be a gripper. The end effector 432 may be sterile in order to remove each item from the packages, and then arrange each item on the sterile tray 156. The end effector 432 may be removable and autoclavable.

FIG. 4C depicts a block diagram of the setup robot 400 depicted in FIG. 4B, in accordance with embodiments of the present disclosure.

The control system 420 is coupled to the drive system 402, the proximity sensors 404, the forward-looking camera 407, the line-following camera 408, the robotic arm 410, the end effectors 412, 413, and the end effector camera 414. In certain embodiments, the control system 420 may include a processor 422 coupled to a memory 424, a WiFi transceiver 426, and a GPS receiver 428. The WiFi transceiver 426 is coupled to the WiFi antenna 405, and the GPS receiver 428 is coupled to the GPS antenna 406. The WiFi antenna 405 and the GPS antenna 406 may be mounted to an external surface of the chassis 401 to improve signal transmission and reception. In some embodiments, the WiFi transceiver 426 and the GPS receiver 428 may incorporate antennae within their respective form factors.

In certain embodiments, the control system 420 may be coupled to the second robotic arm 430, end effector 432, and camera 434.

In certain embodiments, the control system 420 may navigate to the second location in the second operating room 150 based on sensor data, open the packages on the storage table 157, remove the items from the packages, and deposit the items on the sterile tray 156 on the sterile table 155. After the ophthalmic procedure in the first operating room 130 is completed and the packages needed for the next ophthalmic procedure in the first operating room 130 have been delivered to the storage table 137, the control system 420 may navigate to the first location in the first operating room 130 based on sensor data, open the packages on the storage table 137, remove the items from the packages, and deposit the items on the sterile tray 136 on the sterile table 135. The sensor data may include proximity data from the proximity sensors 404, image data from the forward-looking camera 407, image data from the line-following camera 408, and position data from the GPS receiver 428.

A map of the hallway 110, the first operating room 130, and the second operating room 150 may be stored in the memory 424 of the control system 420. The map may include, inter alia, the dimensions of the hallway 110, the first operating room 130, and the second operating room 150, the navigation lines, and the location and dimensions of the obstacles in the first operating room 130 and the second operating room 150, such as instruments, consoles, tables, stations, etc.

The control system 420 (such as the processor 422) may process the sensor data and execute one or more models (such as machine learning models, etc.), etc., to determine a current location based on the sensor data and the map, determine a desired location based on the map, navigate to the desired location based on the sensor data and the map, and then perform the appropriate function.

For example, the setup robot 400 may be located in the hallway 110 (the current location), and the desired location may be the second location that is adjacent to the sterile table 155 and the storage table 157 in the second operating room 150. The control system 420 may then navigate from the hallway 110 to the second location in the second operating room 150. In another example, the setup robot 400 may be located at the second location in the second operating room 150 (the current location), and the desired location may be the first location that is adjacent to the sterile table 135 and the storage table 137 in the first operating room 130. The control system 420 may then navigate from the second location in the second operating room 150 to the first location in the first operating room 130. In a further example, the setup robot 400 may be located at the first location in the first operating room 130 (the current location), and the desired location may be a central location in the hallway 110. The control system 420 may then navigate from the first location in the first operating room 130 to the hallway 110.

In certain embodiments, the control computer 172 may transmit an “open packages” command to the setup robot 400 over a wireless communication link. The open packages command may be directed to the first operating room 130 (or the second operating room 150), and include an item list that identifies the packages that are to be opened and the items that are to be deposited on the sterile tray.

In response to receiving the open packages command, the control system 420 may navigate to the first location in the first operating room 130 (or the second location in the second operating room 150) based on sensor data, open the packages on the storage table 137 (or the storage table 157), remove the items from the packages, and deposit the items on the sterile tray 136 on the sterile table 135 (or the sterile tray 156 on the sterile table 155). After the items have been deposited, the control system 420 may transmit data, such as an open packages response, etc., to the control computer 172 that indicates that the items in the packages have been deposited. In response, the control computer 172 may transmit another open packages command to the setup robot 400 over the wireless communication link. Alternatively, the control computer 172 may transmit a “relocate” command to the setup robot 400 over the wireless communication link. The relocate command may instruct the setup robot 400 to move to another location in the first operating room 130, a location in the second operating room 150, a location in the hallway 110, etc.

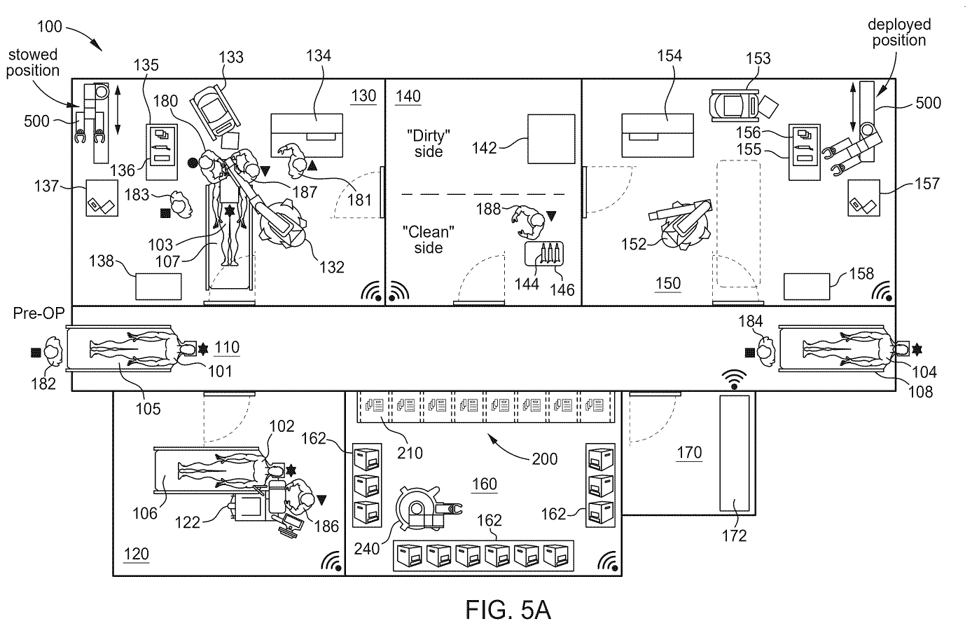

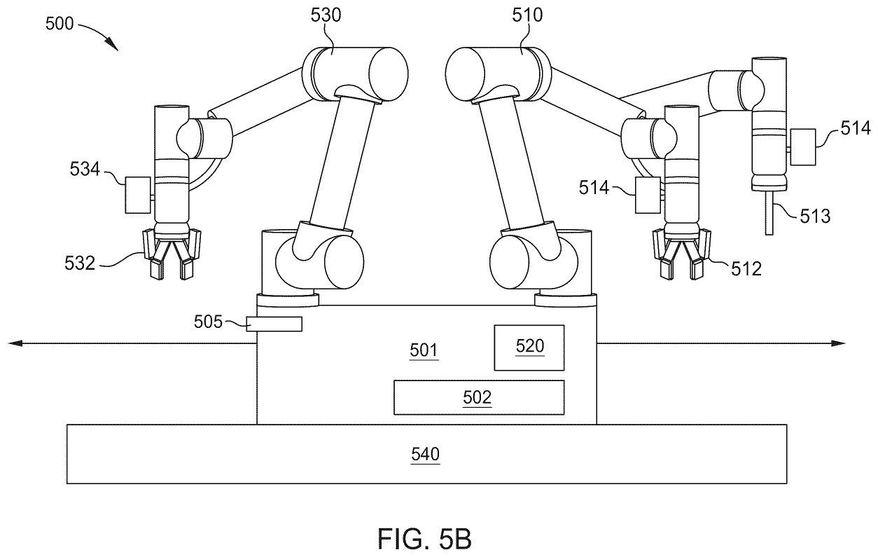

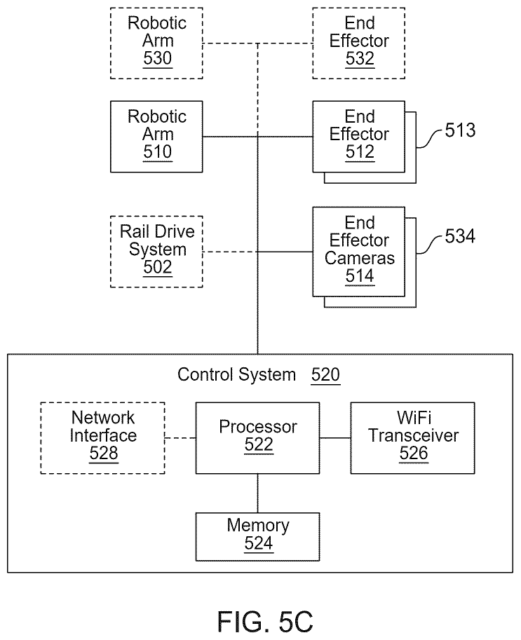

FIG. 5A depicts another schematic diagram of the surgical facility 100 for ophthalmic procedures, in accordance with embodiments of the present disclosure.

In certain embodiments, the surgical facility 100 may include a pre-operation (pre-op) room or holding area, a post-operation (post-op) room or holding area, a hallway 110, an eye imaging room 120, a first operating room 130, a sterilization room 140, a second operating room 150, a storage room 160, and a computer room 170, as described above with respect to FIG. 1. The surgical facility 100 also includes a setup robot 500 in the first operating room 130, and a setup robot 500 in the second operating room 150.

The setup robot 500 may translate along a rail between a stowed position when not in use, and a deployed position when preparing the items that are needed for the next ophthalmic procedure. The setup robot 500 in the first operating room 130 is depicted in the stowed position, while the setup robot 500 in the second operating room 150 is depicted in the deployed position. The deployed position places the setup robot 500 adjacent to the sterile table 155 (or the sterile table 135) and the storage table 157 (or the storage table 137).

Advantageously, the setup robot 500 may reduce or eliminate the need for one nurse and one scrub technician, such as the nurse 185 and the scrub technician 189 stationed in the second operating room 150. The nurse 183 and the scrub technician 187 would then rotate between the first operating room 130 and the second operating room 150 with the surgeon 180 and the anesthesiologist 181.

As discussed above, the packages needed for the next ophthalmic procedure may include one or more IOL packages 164, one or more surgical packages 166, and a sterile package for a reusable instrument 144. As depicted in FIG. 5A, the packages needed for the next ophthalmic procedure in the second operating room 150 have already been delivered to the storage table 157. In certain embodiments, in response to receiving the next procedure command from the control computer 172, a delivery robot 300 may retrieve a bin 230 from a compartment 210 in the dispenser 200, retrieve a sterile package from the sterilization room 140 (if needed) and place it in the bin 230, and deliver the contents of the bin 230 to the storage table 157 (as depicted FIG. 3A). In other embodiments, the nurse 183 may retrieve the packages needed for the next ophthalmic procedure from the second staging table 114 (as depicted FIG. 1), and deposit them on the storage table 157.

After the packages are delivered to the storage table 157 in the second operating room 150, the setup robot 500 may open the packages, remove the items from the packages, and then deposit the items on the sterile tray 156 on the sterile table 155.

In certain embodiments, the setup robot 500 may cut open each package using a robotic arm with non-sterile end effectors, and then invert each package over the sterile tray 156 to remove the items and deposit them on the sterile tray 156. In other embodiments, the setup robot 500 may cut open each package using a first robotic arm with non-sterile end effectors, remove each item from the packages using a second robotic arm with a sterile end effector, and then arrange each item on the sterile tray 156 using the second robotic arm and the sterile end effector.

FIG. 5B depicts a diagram of a setup robot 500, in accordance with embodiments of the present disclosure.

In certain embodiments, the setup robot 500 may include, inter alia, a chassis 501, a drive system 502 that includes a motor to translate the chassis 501 along a rail 540, a WiFi antenna 505, a robotic arm 510 including end effectors 512, 513 and end effector cameras 514, and a control system 520.

The end effector 512 may be a gripper. Generally, the end effector 513 is configured to open the IOL, surgical, and sterile packages, and may be removable and autoclavable. In certain embodiments, the end effector 513 may be a cutting tool, such as a knife, a circular saw blade, etc., that is configured to cut through the material of the IOL, surgical, and sterile packages to create an opening in the package. In other embodiments, the end effector 513 may be a vacuum tool, a heat plate, etc.

In certain embodiments, the setup robot 500 may include a second robotic arm 530 including an end effector 532 and a camera 534. The end effector 532 may be a gripper. The end effector 532 may be sterile in order to remove each item from the packages, and then arrange each item on the sterile tray 156. The end effector 532 may be removable and autoclavable.

FIG. 5C depicts a block diagram of the setup robot 500 depicted in FIG. 5B, in accordance with embodiments of the present disclosure.

The control system 520 is coupled to the drive system 502, the robotic arm 510, the end effectors 512, 513, and the end effector camera 514. In certain embodiments, the control system 520 may include a processor 522 coupled to a memory 524, and a WiFi transceiver 526. In some embodiments, the control system 520 may also include a network interface 528 that is coupled to a wired network (such as Ethernet, etc.). The WiFi transceiver 526 is coupled to the WiFi antenna 505. In certain embodiments, the control system 520 may be coupled to the second robotic arm 530, an end effector 532, and camera 534.

For the setup robot 500 in the first operating room 130, the control system 520 may open the packages on the storage table 137, remove the items from the packages, and then deposit the items on the sterile tray 136 on the sterile table 135. Similarly, for the setup robot 500 in the second operating room 150, the control system 520 may open the packages on the storage table 157, remove the items from the packages, and then deposit the items on the sterile tray 156 on the sterile table 155.

In certain embodiments, the control computer 172 may transmit a “deploy” command to the setup robot 500 over the wireless communication link to translate to the deployed position.

In certain embodiments, the control computer 172 may transmit an “open packages” command to the setup robot 500 over a wireless (or wired) communication link. The open packages command may be directed to the setup robot 500 in the first operating room 130 (or to the setup robot 500 in the first operating room 130), and include an item list that identifies the packages that are to be opened and the items that are to be deposited on the sterile tray.

In response to receiving the open packages command, the control system 520 may translate from the stowed position to the deployed position, open the packages on the storage table 137 (or the storage table 157), remove the items from the packages, and then deposit the items on the sterile tray 136 on the sterile table 135 (or the sterile tray 156 on the sterile table 155). After the items have been deposited, the control system 520 may transmit data, such as an open packages response, etc., to the control computer 172 that indicates that the items in the packages have been deposited. In response, the control computer 172 may transmit a “relocate” command to the setup robot 500 over the wireless communication link to translate back to the stowed position, etc.

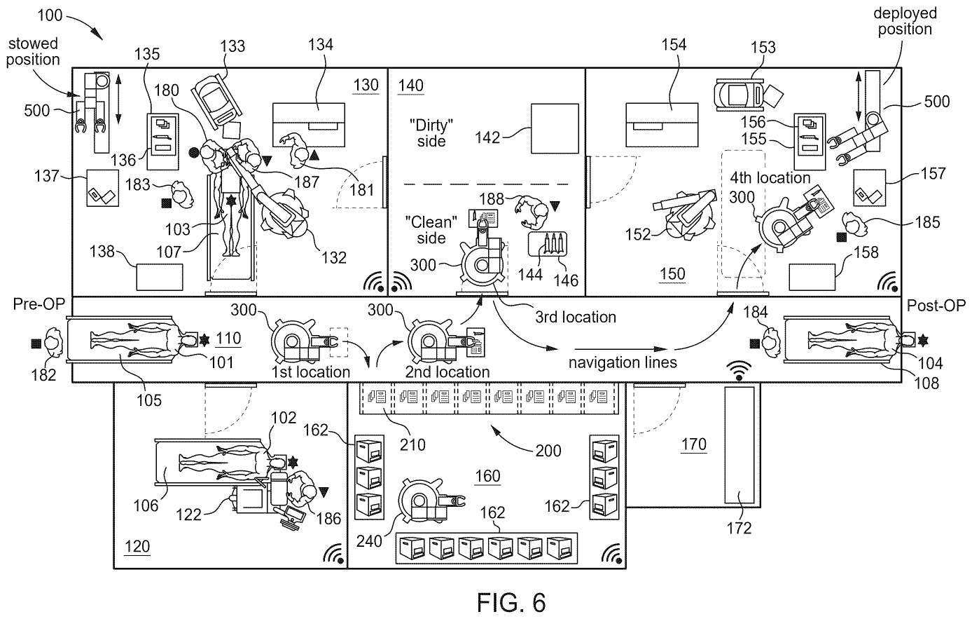

FIG. 6 depicts another schematic diagram of the surgical facility 100 for ophthalmic procedures, in accordance with embodiments of the present disclosure.

In certain embodiments, the surgical facility 100 may include a pre-operation (pre-op) room or holding area, a post-operation (post-op) room or holding area, a hallway 110, an eye imaging room 120, a first operating room 130, a sterilization room 140, a second operating room 150, a storage room 160, and a computer room 170, as described above with respect to FIG. 1. The staging tables 112, 114 in the hallway 110 are not needed.

The surgical facility 100 also includes a storage robot 240 in the storage room 160, a delivery robot 300, a setup robot 500 in the first operating room 130, and a setup robot 500 in the second operating room 150. Generally, the control computer 172 coordinates the activities of the storage robot 240, the delivery robot 300, and the setup robots 500 according to the principles described above.

In other embodiments, the surgical facility 100 includes a setup robot 400 in place of the setup robots 500. Generally, the control computer 172 coordinates the activities of the storage robot 240, the delivery robot 300, and the setup robot 400 according to the principles described above.

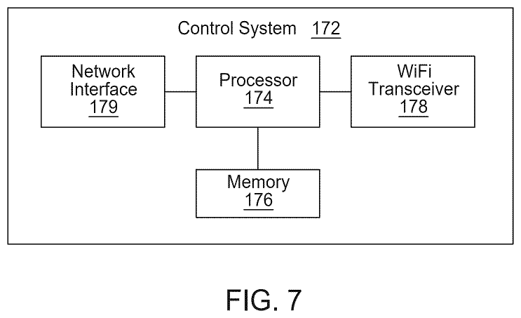

FIG. 7 depicts a block diagram for a control computer 172 for the surgical facility 100, in accordance with embodiments of the present disclosure.

In certain embodiments, the control computer 172 may include a processor 174 coupled to a memory 176, a WiFi transceiver 178, and a network interface 179. The WiFi transceiver 178 is coupled to a WiFi antenna, and the network interface 179 may be coupled to a wired network (such as Ethernet, etc.).

The processor 174 may be configured to generate and transmit various commands, such as the dispenser command, the compartment command, the next procedure command, the open packages command, the relocate command, etc., and to receive various responses, such as the dispenser response, the compartment response, the next procedure response, the open packages response, the error response, etc. The processor 174 may also be configured to track the inventory of the IOL and surgical packages placed in the bins 230 of the dispenser 200 through the dispenser and compartment responses, and monitor error through the error response.

### Example Embodiments

In certain embodiments, a robotic system for a surgical facility comprises a dispenser, a delivery robot, and a first setup robot located in a first operating room. The delivery robot comprises a drive system, sensors configured to generate sensor data, a robotic arm comprising a camera and a first gripper, and a control system. The control system is configured to navigate to the dispenser based on the sensor data, retrieve a first bin from the dispenser using the first gripper, the first bin including a first surgical package, navigate to a first storage table in the first operating room based on the sensor data, and deliver the first surgical package to the first storage table using the first gripper. The first setup robot comprises a robotic arm comprising a camera, a first end effector configured to hold and manipulate a package, and a second end effector configured to open the package, and a control system. The control system is configured to open the first surgical package located on the first storage table using the first and second end effectors, remove items from the first surgical package using the first end effector, and deposit the items on a first sterile tray in the first operating room using the first end effector.

In certain embodiments, a delivery robot for a surgical facility comprises a drive system; sensors configured to generate sensor data; a robotic arm comprising a camera and a gripper; and a control system. The control system is configured to navigate to a dispenser based on the sensor data, retrieve a bin from the dispenser using the gripper, the bin including a surgical package, navigate to a first storage table in an operating room based on the sensor data, and deliver the surgical package to the first storage table using the gripper.

In certain embodiments, a setup robot for a surgical facility comprises a first robotic arm comprising a camera, a first end effector configured to hold and manipulate a package, and a second end effector configured to open the package; and a control system. The control system is configured to open one or more packages located on a storage table in an operating room using the first and second end effectors, the one or more packages comprising a surgical package, an intraocular lens (IOL) package, or an instrument package, remove items from the one or more packages using the first end effector, and deposit the items on a sterile tray in the operating room using the first end effector.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A robotic system for a surgical facility, the robotic system comprising:
a dispenser;
a delivery robot comprising:
a drive system,
sensors configured to generate sensor data,
a robotic arm comprising a camera and a first gripper, and
a control system configured to:
navigate to the dispenser based on the sensor data,
retrieve a first bin from the dispenser using the first gripper, the first bin including a first surgical package,
navigate to a first storage table in a first operating room based on the sensor data, and
deliver the first surgical package to the first storage table using the first gripper; and
a first setup robot located in the first operating room, the first setup robot comprising:
a robotic arm comprising a camera, a first end effector configured to hold and manipulate a package, and a second end effector configured to open the package, and
a control system configured to:
open the first surgical package located on the first storage table using the first and second end effectors,
remove items from the first surgical package using the first end effector, and
deposit the items on a first sterile tray in the first operating room using the first end effector.

2. The robotic system of claim 1, wherein:
the first bin further includes an intraocular lens (IOL) package; and
the control system of the delivery robot is further configured to:
navigate to a sterilization room based on the sensor data,
retrieve an instrument package from a sterilization storage table in the sterilization room,
deposit the instrument package in the first bin,
navigate to the first storage table in the first operating room based on the sensor data, and
deliver the IOL package and the instrument package to the first storage table using the first gripper.

3. The robotic system of claim 1, wherein the drive system of the delivery robot comprises a plurality of articulated wheels, and the sensors of the delivery robot comprise one or more cameras, one or more proximity sensors, and a global positioning system (GPS) receiver.

4. The robotic system of claim 1, wherein:
the first setup robot further comprises:
a drive system comprising a plurality of articulated wheels, and
sensors configured to generate sensor data, the sensors comprising one or more cameras, one or more proximity sensors, and a GPS receiver; and
the control system of the first setup robot is further configured to navigate at least within the first operating room based on the sensor data.

5. The robotic system of claim 1, wherein the control system of the delivery robot is further configured to:
navigate to the dispenser based on the sensor data;
retrieve a second bin from the dispenser using the first gripper, the second bin including a second surgical package;
navigate to a second storage table in a second operating room based on the sensor data; and
deliver the second surgical package to the second storage table using the first gripper.

6. The robotic system of claim 5, further comprising:
a second setup robot located in the second operating room, the second setup robot comprising:
at least one robotic arm comprising a camera, a third end effector configured to hold and manipulate a package, and a fourth end effector configured to open the package, and
a control system configured to:
open the second surgical package located on the second storage table using the third and fourth end effectors,
remove items from the second surgical package using the third end effector, and
deposit the items on a second sterile tray in the second operating room using the third end effector.

7. The robotic system of claim 1, wherein:
the dispenser comprises a front side, a back side, and a plurality of compartments;
each compartment is configured to store a bin; and
each compartment is accessible from the front side and the back side.

8. The robotic system of claim 7, wherein the delivery robot navigates to the front side of the dispenser, and retrieves the first bin from one of the compartments of the dispenser.

9. The robotic system of claim 7, further comprising:
a storage robot located in a storage room adjacent to the dispenser, the storage robot comprising:
a drive system,
sensors configured to generate sensor data,
a robotic arm comprising a camera and a second gripper, and
a control system configured to:
navigate to a storage rack within the storage room based on the sensor data,
retrieve one or more surgical packages from the storage rack using the second gripper,
navigate to the back side of the dispenser based on the sensor data, and
deliver the surgical packages to a bin in one of the compartments of the dispenser using the second gripper.

10. The robotic system of claim 9, wherein the drive system of the storage robot includes a plurality of articulated wheels, and the sensors of the storage robot include one or more cameras, one or more proximity sensors, and a GPS receiver.

11. The robotic system of claim 1, further comprising:
a control computer comprising a processor, a memory, and a wireless transceiver configured to create a wireless communication link with the delivery robot and the first setup robot,
wherein the control system of the delivery robot further comprises a wireless transceiver,
wherein the control system of the first setup robot further comprises a wireless transceiver, and
wherein the processor of the control computer is configured to send commands to, and receive data from, the delivery robot and the first setup robot over the wireless communication link.

12. The robotic system of claim 11, further comprising:
a storage robot located in a storage room adjacent to the dispenser,
wherein the processor of the control computer is further configured to send commands to, and receive data from, the storage robot over the wireless communication link.

13. A delivery robot for a surgical facility, the delivery robot comprising:
a drive system;
sensors configured to generate sensor data;
a robotic arm comprising a camera and a gripper; and
a control system configured to:
navigate to a dispenser based on the sensor data,
retrieve a bin from the dispenser using the gripper, the bin including a surgical package,
navigate to a first storage table in an operating room based on the sensor data, and
deliver the surgical package to the first storage table using the gripper.

14. The delivery robot of claim 13, wherein the drive system comprises a plurality of articulated wheels, and the sensors comprise one or more cameras, one or more proximity sensors, and a GPS receiver.

15. The delivery robot of claim 13, wherein:
the bin further includes an intraocular lens (IOL) package; and
the control system is further configured to:
navigate to a second storage table in a sterilization room based on the sensor data,
retrieve an instrument package from the second storage table,
deposit the instrument package in the bin,
navigate to the first storage table in the operating room based on the sensor data, and
deliver the IOL package and the instrument package to the first storage table using the gripper.

16. The delivery robot of claim 13, wherein the control system includes a wireless transceiver configured to receive commands from, and send data to, a control computer over a wireless communication link.

17. A setup robot for a surgical facility, the setup robot comprising:
a first robotic arm comprising a camera, a first end effector configured to hold and manipulate a package, and a second end effector configured to open the package; and
a control system configured to:
open one or more packages located on a storage table in an operating room using the first and second end effectors, the one or more packages comprising a surgical package, an intraocular lens (IOL) package, or an instrument package,
remove items from the one or more packages using the first end effector, and
deposit the items on a sterile tray in the operating room using the first end effector.

18. The setup robot of claim 17, further comprising:
a second robotic arm comprising a camera and a sterile gripper,
wherein the control system is further configured to:
remove items from the one or more packages using the sterile gripper,
deposit the items on the sterile tray in the operating room using the sterile gripper, and
arrange the items on the sterile tray using the sterile gripper.

19. The setup robot of claim 17, further comprising:
a drive system comprising a plurality of articulated wheels; and
sensors configured to generate sensor data, the sensors comprising one or more cameras, one or more proximity sensors, and a GPS receiver,
wherein the control system is further configured to navigate at least within the operating room based on the sensor data.

20. The setup robot of claim 17, further comprising:
a drive system comprising a motor coupled to a rail,
wherein the control system is further configured to translate the first robotic arm along the rail using the motor.
